(19) United States
(12) Patent Application Publication
(10) Pub. No.: US 2017/0265722 A1
(43) Pub. Date: Sep. 21, 2017

(54) ASSISTANCE TOOL AND ENDOSCOPE SYSTEM

(71) Applicant: OLYMPUS CORPORATION, Tokyo (JP)

(72) Inventor: Koji YAMAYA, Hachioji-shi (JP)

(73) Assignee: OLYMPUS CORPORATION, Tokyo (JP)

(21) Appl. No.: 15/615,992

(22) Filed: Jun. 7, 2017

Related U.S. Application Data
(63) Continuation of application No. PCT/JP2015/080773, filed on Oct. 30, 2015.

(30) Foreign Application Priority Data
Dec. 10, 2014 (JP) 2014-250215

(51) Int. Cl. A61B 1/00; A61B 1/05

(52) U.S. Cl. CPC A61B 1/00112; A61B 1/053; A61B 1/00147; A61B 1/005

(57) ABSTRACT

An assistance tool that assists in moving a treatment device is provided. The assistance tool includes a flexible tube in which an insertion path is formed to insert the treatment device that is inserted into a channel in an insertion portion of an endoscope, a tube member that is coupled to the flexible tube and has a curved portion halfway of an overall length and in which an insertion path for the treatment device is formed, a coupling portion that is coupled to the tube member and is formed to couple the channel to the insertion path, and a holding portion that is attached to the flexible tube and holds the insertion portion or an operation portion that is coupled to the insertion portion.

### CROSS-REFERENCE TO RELATED APPLICATIONS

This application is a Continuation Application of PCT Application No. PCT/JP2015/080773, filed Oct. 30, 2015 and based upon and claiming the benefit of priority from prior the Japanese Patent Application No. 2014-250215, filed Dec. 10, 2014, the entire contents of all of which are incorporated herein by references.

### BACKGROUND OF THE INVENTION

1. Field of the Invention

The present invention relates to an assistance tool that is attached to an endoscope and a treatment device to assist in moving the treatment device, and an endoscope system having such an assistance tool.

2. Description of the Related Art

There is an endoscopic treatment device insertion tool that is used with an endoscope to insert a treatment device into a channel of the endoscope. The endoscopic treatment device insertion tool described in International Publication No. 2013/065509 has a guide tube, a hand operation portion coupled to the proximal end portion of the guide tube, a window formed along the axial direction of the hand operation portion, and a fixing member slidably provided along the window and fixing a treatment device. A surgeon can advance and retreat the treatment device within the range of the window by operating the fixing member.

### BRIEF SUMMARY OF THE INVENTION

An assistance tool that assists in moving a treatment device according to an aspect of the present invention comprises a flexible tube in which an insertion path is formed to insert the treatment device that is inserted into a channel in an insertion portion of an endoscope, a tube member that is coupled to the flexible tube and has a curved portion halfway of an overall length and in which an insertion path for the treatment device is formed by a rigid tube, a coupling portion that is coupled to the tube member and is formed to couple the channel to the insertion path of the tube member, and a holding portion that is attached to the flexible tube and holds the insertion portion of the endoscope or an operation portion of the endoscope that is coupled to the insertion portion.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram illustrating an overall configuration of an endoscope system according to a first embodiment.

FIG. 2 is a schematic diagram illustrating a distal end surface of a distal end constituent portion of an insertion portion of an endoscope of the endoscope system shown in FIG. 1.

FIG. 3A is a schematic diagram illustrating an assistance tool that assists in moving of a treatment device of the endoscope system shown in FIG. 1.

FIG. 3B is a sectional view taken along line 3B-3B shown in FIG. 3A.

FIG. 4 is a schematic diagram illustrating a tube member of the assistance tool that assists in moving of a treatment device of an endoscope system according to a second embodiment.

FIG. 5 is a schematic diagram illustrating an overall configuration of an endoscope system according to a third embodiment.

FIG. 6A is a schematic diagram illustrating an assistance tool that assists in moving of a treatment device of the endoscope system shown in FIG. 5.

FIG. 6B is a schematic diagram illustrating a holding portion of the assistance tool that assists in moving of the treatment device shown in FIG. 6A, viewed from the direction of arrow 6B.

### DETAILED DESCRIPTION

Hereinafter, embodiments for implementing the present invention will be explained with reference to the drawings.

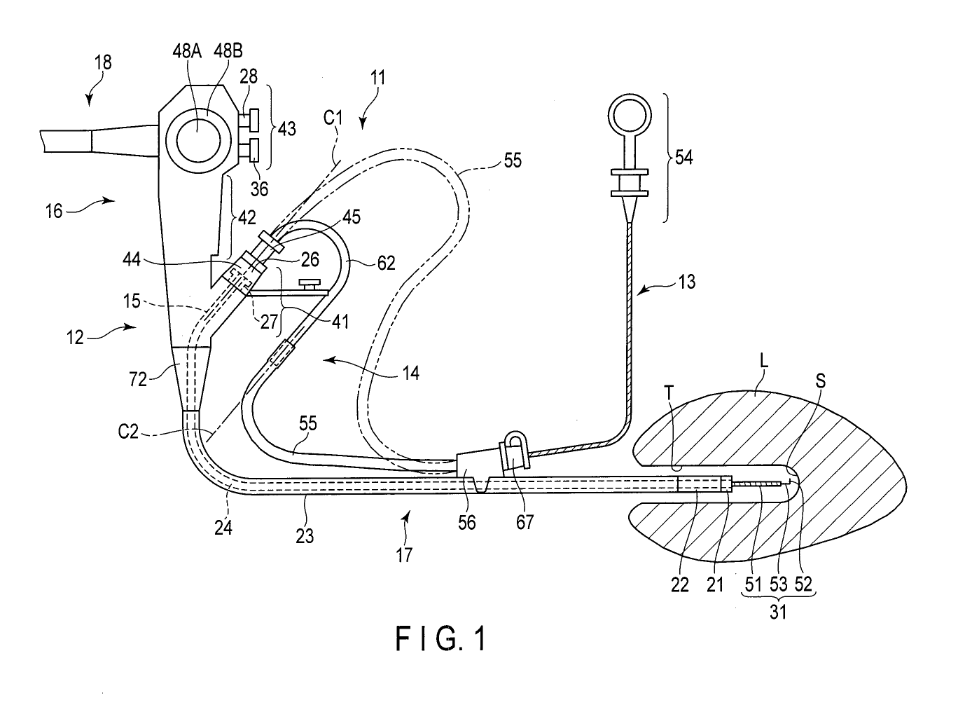

An endoscope system according to the first embodiment will be explained using FIG. 1 to FIG. 3B. The endoscope system 11 (an endoscope apparatus) according to the present embodiment includes an endoscope 12, a treatment device 13, and an assistance tool 14 (an advance and retreat assistance tool) that assists in moving the treatment device. The treatment device 13 can be moved through the treatment device insertion channel 15, which will be described later, of the endoscope 12. The assistance tool 14 that assists in moving of the treatment device is detachably held at an operation portion 16 of the endoscope 12. The assistance tool 14 is used to assist in moving (advancing/retreating and rotating) the treatment device 13 through the treatment device insertion channel 15. In other words, the assistance tool 14 assists a surgeon to move the treatment device 13 extending from one end of the assistance tool 14 through the endoscope 12.

The configuration of the endoscope 12 is briefly explained with reference to FIG. 1. A publicly-known endoscope having a treatment device insertion channel 15 should be used as the endoscope 12 as needed. As shown in FIG. 1, the endoscope 12 includes an insertion portion 17 that is thin and elongated to be inserted into a body cavity, an operation portion 16 provided adjacently to the proximal end portion of the insertion portion 17, and a universal cord 18 extending from the side portion of the operation portion 16.

The insertion portion 17 includes, from the distal end side to the proximal end side, a rigid distal end constituent portion 21, a bending portion 22 having a freely-bendable bending tube in which a plurality of bend pieces, which are not shown in the drawings, are adjacently provided, and a flexible tube portion 23 that can be flexibly bent by an external force, in this order.

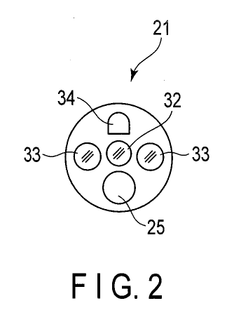

In the inside of the insertion portion 17, a channel tube 24 (a treatment device insertion channel tube) that constitutes the treatment device insertion channel 15 is arranged. The distal end of the channel tube 24 is coupled to a distal end side opening portion 25, which will be described later, of the distal end constituent portion 21 (see FIG. 2). The channel tube 24 is preferably bifurcated in the inside of the operation portion 16, for example, as is publicly known. A first proximal end of the channel tube 24 is coupled to the hand-side opening portion 26 (a first coupling base 27), which will be described later. A second proximal end (not shown) of the channel tube 24 is coupled to a suction button 28, which will be described later, of a suction mechanism. In the treatment device insertion channel 15, a space between the distal end side opening 25 of the distal end constituent portion 21 and the bifurcated portion of the channel tube 24 is formed to be a suction tube and an insertion path into which an insertion body 31 of the treatment device 13 is inserted. In the endoscope 12 according to the present embodiment, a suction mechanism is not always necessary.

As shown in FIG. 2, the distal end constituent portion 21 includes a distal end side opening portion 25, an observation window 32, preferably two (or one) illumination windows 33, and a nozzle 34. As shown in FIG. 1, the distal end side opening portion 25 is coupled to and communicates with the distal end of the channel tube 24 to form a distal end portion of the treatment device insertion channel 15. The observation window 32 constitutes a distal end portion of an observation optical system for observing an area to be observed. The illumination window 33 constitutes a distal end portion of an illumination optical system that illuminates an area to be observed. The nozzle 34 can exhaust gas or liquid from an exhaust port, which is not shown in the drawings, directed toward the observation window 32. The exhaustion of gas and liquid is operated by a gas/water feeding button 36, which will be described later.

As shown in FIG. 1, the operation portion 16 has, from its distal end side to the proximal end side, a treatment device introducing portion 41, a grip portion 42, and an operation portion main body 43, in this order. Preferably, the treatment device introducing portion 41, the grip portion 42, and the operation portion main body 43 are integrally formed.

The treatment device introducing portion 41 includes a hand-side opening portion 26 (a first coupling base 27) to which the first proximal end of the channel tube 24 is coupled. An assistance tool plug 44 is attached at the hand-side opening portion 26 of the treatment device introducing portion 41. The assistance tool plug 44 has a valve structure that prevents liquid flowing in the proximal end side through the channel tube 24 from leaking to the outside of the endoscope 12 while the insertion body 31, which will be described later, of the treatment device 13 and a later-described coupling portion 45 of the assistance tool 14 are being inserted. The assistance tool plug 44 can serve as a brake that prevents the treatment device 13 from being moved unintentionally. As the assistance tool plug 44, a plug similar to a publicly-known forceps plug may be used.

The grip portion 42 is a portion gripped by a surgeon with one of their hands, for example, their left hand. The operation portion main body 43 can operate the bend of the bending portion 22. The universal cord 18 is extended from the side portion of the operation portion main body 43.

The operation portion main body 43 includes bend operation knobs 48A and 48B, a as/water feeding button 36, and a suction button 28. The bend operation knobs 48A and 48B can remotely perform a bend operation on the bending portion 22. Usually, a surgeon operates the bend operation knobs 48A and 48B with their left thumb, etc. while holding the grip portion 42 in their left palm. Since the gas/water feeding mechanism including the gas/water feeding button 36 and the suction mechanism including the suction button 28 are publicly known, descriptions thereof are omitted herein.

The treatment device 13 includes an insertion body 31 that can be inserted into the treatment device insertion channel 15 of the endoscope 12, and a base portion 54 (a hand-side operation portion) arranged at the proximal end of the insertion body 31. The insertion body 31 may be elongated, for example, multiple times longer than the overall length of the treatment device insertion channel 15. Furthermore, the distal end of the insertion body 31 may be put into and taken out of the distal end constituent portion 21 of the insertion portion 17 through the treatment device insertion channel 15 of the endoscope 12. The insertion body 31 generally has a diameter of 1 to 2 mm, for example.

The insertion body 31 includes a sheath 51 and a wire 53 having an end effector 52 at its distal end. The sheath 51 may be simply made of a resin-made tube having insulation properties, or may be a coil sheath, for example, and the sheath 51 is selected depending on the end effector 52. The wire 53 is flexible.

For the end effector 52, an end effector in approximately an ‘L’ shape, a snare-shaped end effector, or a basket-shaped effector, may be used as needed. The end effector 52 can perform a process as needed, using high-frequency energy, on living tissue present between the end effector 52 and a return pad, which is not shown in the drawings, attached to a patient.

The base portion 54 has a publicly-known slider mechanism. The wire 53 can move in the axial direction thereof with respect to the sheath 51 by operating a slider mechanism in the base portion 54. For this reason, by the slider mechanism, the wire 53 can advance and retreat along the axial direction of the insertion body 31 with respect to the sheath 51. The insertion body 31, i.e., the sheath 51 and the wire 53, held by the surgeon can be rotated forward and backward or rotated integrally with the insertion body 31 in one direction around the axis of the insertion body 31.

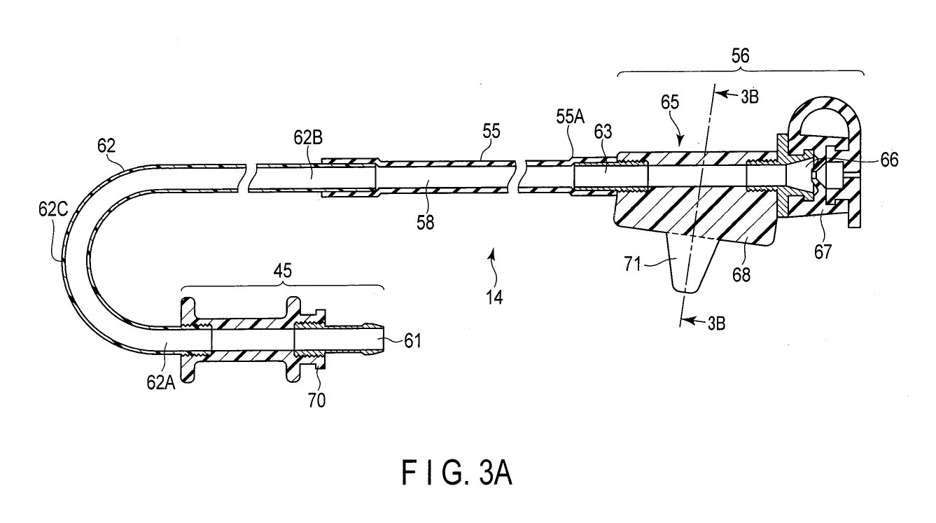

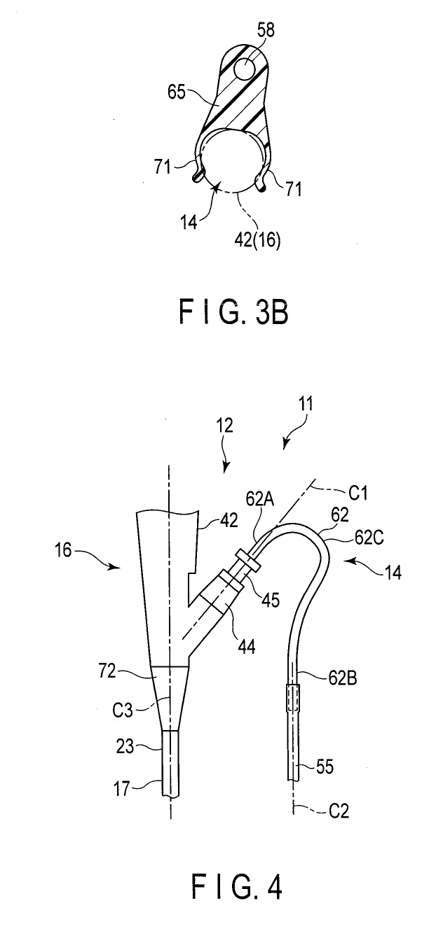

The assistance tool 14 that is used to assist in moving the treatment device 13 through the treatment device insertion channel 15 of the endoscope 12 is explained with reference to FIG. 1 and FIG. 3B. The assistance tool 14 (an advance/retreat assistance tool) includes an elongated flexible tube 55, a tube member 62 (a rigid tube) coupled to the flexible tube 55, a coupling portion 45 that detachably couples the proximal end (a coupling end) side of the tube member 62 to the hand-side opening portion 26 of the endoscope 12, and a holding portion 56 that is provided on the distal end (a moving end) side of the flexible tube 55 to hold the outer periphery of the insertion portion 17 of the endoscope 12. The tube member 62 is located closer to the side of the operation portion 16 of the endoscope 12 than the flexible tube 55 is.

For the flexible tube 55, it is preferable to use a tube which is easily bendable and difficult to break, and has excellent slidability for the insertion body 31 of the treatment device 13. For the flexible tube 55, for example, a fluorine-based resin tube, a fluorine-based resin tube in which a blade is inserted to increase bend resistance, a blade-inserted polyurethane-based resin tube coated with a fluorine-based resin in the insertion path 58 inside the flexible tube 55 to make it easy for the insertion body 31 of the treatment device 13 to advance or retreat along the axial direction, and the like, may be used. Although the flexible tube 55 is formed shorter than the overall length of the insertion body 31 of the treatment device 13, the length of the order of 400 mm is preferable in terms of usage.

As shown in FIGS. 3A and 3B, the flexible tube 55 has a distal end side base 63 at its distal end side. The distal end side base 63 is coupled to the holding portion 56. The holding portion 56 is attached to the terminal portion 55A, which is located on the opposite side of the terminal portion that is coupled to the tube member 62 of the flexible tube 55.

The tube member 62 is made from a cylindrical tube made of a resin or a metal, for example. The tube member 62 has strength to the extent that it is not deformed under a certain level of externally-applied power, and thus, the tube member 62 can be regarded as almost a rigid body. In other words, the tube member 62 has higher rigidity or is harder than the flexible tube 55. The tube member 62 includes an end 62A coupled to the coupling portion 45, an other end 62B coupled to the flexible tube 55, and a curved portion 62C provided in between in the longitudinal direction. As shown in FIG. 1, the second central axis C2 of the end 62B extends in a direction along the direction of the first central axis C1 of the end 62A.

As shown in FIGS. 3A and 3B, the tube member 62 is formed by bending it into an overall ‘U’ or ‘J’ shape. The radius of curvature of the curved portion of the tube member 62 is appropriately determined to be of a value as small as possible within a range that allows the treatment device 13 to advance and retreat with no problems. In other words, in the present embodiment, since the radius of curvature of the tube member 62 is formed as minimal as possible, the tube member 62 does not interfere when handling the operation portion 16, as the tube member 62 does not project from the operation portion 16 and the insertion portion 17. The inner surface of the tube member 62 (the insertion path 58) is coated with a fluorine-based resin to provide excellent slidability for the treatment device 13 in the insertion path 58.

The flexible tube 55, the tube member 62, the coupling portion 45, and the holding portion 56 of the assistance tool 14 form the insertion path 58. The insertion path 58 is formed along the overall length of the assistance tool 14, and the insertion body 31 of the treatment device 13 is inserted through the insertion path 58.

The coupling portion 45 is formed so as to couple (connect) the treatment device insertion channel 15 to the insertion path 58 of the tube member 62. The coupling portion 45 has a second coupling base 61 having a proximal end side opening end of the insertion path 58. The second coupling base 61 is coupled to the assistance tool plug 44, while keeping the valve structure of the assistance tool plug 44 open at the proximal end side opening end. In other words, in the present embodiment, the second coupling base 61 of the coupling portion 45 and a projection portion 70 in the proximity thereof are inserted, for engagement, into a recess portion (not shown) in the assistance tool plug 44 made of an elastic material, which is attached to the hand-side opening portion 26 of the endoscope 12. For this reason, the coupling portion 45 of the assistance tool 14 is connected to the treatment device insertion channel 15 of the endoscope 12 via the assistance tool plug 44 (the first coupling base 27) of the hand-side opening portion 26 of the endoscope 12, so that the channel is connected to the insertion path 58. The coupling portion 45 of the assistance tool 14 is detachable from the assistance tool plug 44 attached to the hand-side opening portion 26 of the endoscope 12.

A method of coupling the coupling portion 45 to the hand-side opening portion 26 of the endoscope 12 is not limited to the example of the present embodiment. The coupling portion 45 may be directly fixed to the hand-side opening portion 26 in a detachable manner, without using the assistance tool plug 44 to prevent liquid leakage. More specifically, the coupling portion 45 may be made of an elastic material, such as silicone rubber, not of a resin material, for example, to fix the coupling portion 45 directly to the hand-side opening portion 26 of the endoscope 12 in a watertight manner. Furthermore, the coupling portion 45 may be omitted; instead, the proximal end side of the flexible tube 55 may be directly inserted into the treatment device insertion channel 15 to fix the assistance tool 14 and the endoscope 12.

As shown in FIGS. 3A and 3B, the holding portion 56 includes a holding portion main body 65, a third coupling base 66 attached to the holding portion main body 65, and a treatment device plug 67. The third coupling base 66 has a same mechanism as that of the first coupling base 27 of the endoscope 12. Similar to the above-described assistance tool plug 44, a plug similar to a publicly-known forceps plug may be used as a treatment device plug 67. Thus, due to its valve structure, the treatment device plug 67 can prevent body fluid in the body cavity flowing in the treatment device insertion channel 15 of the endoscope 12 and the insertion path 58 of the assistance tool 14 from leaking out. Furthermore, even when the insertion body 31 of the treatment device is being inserted, the treatment device plug 67 can prevent liquid, such as body fluid in the body cavity flowing in the treatment device insertion channel 15 of the endoscope 12 and the insertion path 58 of the assistance tool 14, from leaking out. The treatment device plug 67 serves as a brake that prevents the treatment device 13 from being moved unintentionally.

The holding portion main body 65 is made of a resin, etc., for example. The holding portion main body 65 is detachable from the insertion portion 17 of the endoscope 12. The holding portion main body 65 includes a base body 68 that forms the proximal end portion of the insertion path 58 and a pair of arms 71 extending from the base body 68. As shown in FIG. 3B, the pair of arms 71 makes an overall ‘C’ shape. The holding portion main body 65 can hold the insertion portion 17 (the flexible tube portion 23) between the arms 71 when the space between the arms is temporarily widened for the flexible tube portion 23 (the insertion portion 17), which is shown in a dashed line, as a result of elastically deforming the arms 71. Thus, the holding portion main body 65 can be easily attached to and detached from the outer surface of the insertion portion 17.

Next, the effects of the endoscope system 11 configured as described above will be explained with reference to FIG. 1. Herein, an example of endoscopic submucosal dissection (ESD) in which a layer under a site of lesion S is filled with a liquid, such as a physiological salt solution, etc., and the margin of the site of lesion S is resected by electrocautery.

The surgeon grips the grip portion 42 of the endoscope 12 with their left hand, and grips the outer surface of the insertion portion 17 with their right hand. The surgeon inserts the distal end constituent portion 21 at the distal end of the insertion portion 17 through the opening of a cavity T of living tissue L toward the site of lesion S, while looking at an observation image displayed on a monitor, which is not shown in the drawings, by the observation optical system of the endoscope 12. At this time, the surgeon operates the bend operation knobs 48A and 48B as needed to perform a push-and-pull operation and a twist operation of the insertion portion 17 with their right hand, while gripping the grip portion 42 of the endoscope 12 with their left hand. And then, the surgeon brings the distal end constituent portion 21 of the insertion portion 17 close to the site of lesion S with an appropriate distance.

While the surgeon is gripping the insertion portion 17 of the endoscope 12 with their right hand and maintaining the position relationship between the distal end surface of the distal end constituent portion 21 of the insertion portion 17 and the site of lesion S, an assistant (another surgeon) attaches the coupling portion 45 at the proximal end of the assistance tool 14 to the hand-side opening portion 26 of the endoscope 12 (the first coupling base 27), and attaches the holding portion 56 at the distal end of the assistance tool 14 to the insertion portion 17 with the arms 71. After that, the assistant inserts the insertion body 31 of the treatment device 13 into the insertion path 58 of the assistance tool 14 and the treatment device insertion channel 15 of the endoscope 12 to arrange the end effector 52 in the proximity of the distal end constituent portion 21 of the insertion portion 17 of the endoscope 12. Thus, preparation before treatment is completed.

The surgeon grips the insertion portion 17 with the palm and second to fourth fingers of their right hand, and pushes and pulls the treatment device 13 by holding the treatment device 13 with the thumb and first finger of their right hand to make fine adjustments, for example, ±10 mm, for the position of the treatment device 13 (the insertion body 31) in the axial direction. In other words, the surgeon can perform delicate techniques of high-frequency incision with, for example, the treatment device 13 using high-frequency energy, by delicately adjusting the length of the projection of the end effector 52 of the treatment device 13 from the distal end constituent portion 21 of the insertion portion 17 of the endoscope 12 with the thumb and first finger of their right hand, while performing a push-and-pull operation and a twist operation of the insertion portion 17, gripping the insertion portion 17 with their right hand.

At this time, since the flexible tube 55 is arranged in the proximity of the insertion portion 17 and the operation portion 16 of the endoscope 12 due to the tube member 62, the flexible tube 55 does not interfere with the surgeon's and operator's work. Since the hard tube member 62 is used, the assistance tool 14 and the treatment device 13 are prevented from being buckled during treatment. As shown by the chain dash line in FIG. 1, if the flexible tube 55 constitutes the overall length from the coupling portion 45 to the holding portion 56, the flexible tube 55 would project at a position distant from the endoscope 12. However, since a rigid tube member 62 is used in the present embodiment, it is possible to make the radius of curvature of the tube member 62 small.

The base portion 54 of the treatment device 13 is usually operated by an assistant who is not the surgeon using the endoscope 12, in accordance with instructions from the surgeon. The start/stop of outputting high-frequency energy is usually performed by the surgeon's operation of, for example, a foot switch.

In ESD treatment, a treatment device 13 is changed to another and inserted into the assistance tool 14 and the treatment device insertion channel 15 of the endoscope 12. The change operation is performed by an assistant (another surgeon) who is not holding the insertion portion 17 of the endoscope 12.

First, the margin of a site of lesion S is marked with the first treatment device 13 (a marking treatment device). Next, the first treatment device 13 is changed to a second treatment device 13 (a local injection treatment device), and a pharmaceutical agent, such as a physiological salt solution, is locally injected into a submucous layer of the site of lesion S. After that, the second treatment device 13 is further changed to a third treatment device 13 (for example, a high-frequency knife), and the mucosa in the margin of the site of lesion S is incised around the marking with the third treatment device 13. Then, the submucous layer of the site of lesion S is dissected, preferably using the same third treatment device 13 (the high-frequency knife). Then, a hemostasis treatment is performed on the site of the dissection using high-frequency energy, etc., and the third treatment device 13 is changed to a fourth treatment device 13 (for example, gripping forceps) to collect the lesion S.

In the present embodiment, since the tube member 62 brings the flexible tube 55 to a position in the proximity of the insertion portion and the operation portion 16 of the endoscope 12, even in a case of using a plurality of treatment devices 13, the flexible tube 55 does not interfere with the surgeon's or operator's work. When the surgeon twists the insertion portion 17 around its axis with their right hand to rotate the insertion portion 17 forward and backward around its axis, their right hand is not removed from the insertion portion 17. Thus, when the marking is made on the margin of the site of lesion S, it is possible to prevent loosing sight of the lesion. Similarly, during the operation of incising and dissecting the site of lesion S along the marking, it is possible to maintain the status of gripping the insertion portion 17 with the right hand. It is possible for the surgeon to delicately adjust the depth of the incision by advancing and retreating the insertion body 31 (the treatment device 13) by the thumb and first finger of their right hand.

Then, the surgeon gives a cue to the assistant to output energy when the incision operation or the dissection operation on the site of lesion S is performed using high-frequency energy, for example. The treatment device plug 67 having a valve structure prevents leakage of blood, etc. from the treatment device plug 67 and the assistance tool 14 when treatment is performed. When the treatment device 13 is changed, or after treatment is finished, the assistant pulls out the insertion body 31 of the treatment device 13 from the treatment device insertion channel 15 and the insertion path 58 of the assistance tool 14. Also at this time, the treatment device plug 67 having a valve structure prevents leakage of blood, etc. The assistance tool 14 is reused after cleaning, disinfecting, and sterilizing.

The assistance tool that assists in moving a treatment device according to the first embodiment comprises the flexible tube 55 in which the insertion path 58 is formed to insert the treatment device 13 that is inserted into a channel in the insertion portion 17 of the endoscope 12, the tube member 62 that is coupled to the flexible tube 55 and has the curved portion 62C halfway of an overall length and in which the insertion path 58 for the treatment device 13 is formed by a rigid tube, the coupling portion 45 that is coupled to the tube member 62 and is formed to couple the channel to the insertion path 58 of the tube member 62, and the holding portion 56 that is attached to the flexible tube 55 and holds the insertion portion 17 of the endoscope 12 or the operation portion 16 of the endoscope 12 that is coupled to the insertion portion 17.

The endoscope system 11 comprises the assistance tool, the endoscope 12 including the insertion portion 17 that is inserted into a body cavity and in which a channel for inserting the treatment device 13 into an inside thereof is provided, and the operation portion 16 that is coupled to the insertion portion 17 and operates the insertion portion 17, and the treatment device 13 that is inserted into the endoscope 12 and is assisted in advancing and retreating by the assistance tool that assists the move of the treatment tool.

Generally speaking, if the flexible tube 55 constitutes the overall length from the coupling portion 45 to the holding portion 56, the radius of curvature of the flexible tube 55 changes not only depending on a material of the flexible tube 55 and a dimension of an inner or outer diameter, but also depending on whether the treatment device 13 is inserted or not. Accordingly, it is difficult to control the radius of curvature of the flexible tube 55 when the flexible tube 55 constitutes the overall length from the coupling portion 45 to the holding portion 56.

According to the foregoing configuration, the use of the tube member 62 can prevent a variation in the radius of curvature of the flexible tube 55. Thus, it is possible to prevent an amount of force required for advancing and retreating the treatment device 13 from becoming variable, and the surgeon can perform treatment with a steady amount, of force. Since the tube member 62 is made as a rigid body having a curved portion 62C on half of the tube member 62, it is possible to arrange the flexible tube 55 in the closer proximity of the endoscope 12 when compared to the case where the coupling portion 45 and the holding portion 56 are coupled only by the flexible tube 55, and it is possible to prevent to the utmost the flexible tube 55 from extending to a location distant from the endoscope 12. Thus, it is possible to prevent the projected flexible tube 55 from interfering with the surgeon's or assistant's work. Accordingly, the surgeon or the operator will not accidentally catch their arm, etc. on the flexible tube 55, and breakage of the flexible tube 55 and damage to the treatment device 13 arranged in the inside of the flexible tube 55 can be prevented.

In this case, the tube member 62 includes the other end 62A coupled to the coupling portion 45 and the end 62B coupled to the flexible tube 55, and in which the central axis C2 of the end 62B extends in the direction of the central axis C1 of the end 62A. According to this configuration, it is possible to arrange the end 62B of the tube member 62 in the proximity of the endoscope 12. Thus, it is possible to prevent to the utmost the flexible tube 55 from extending to a location distant from the endoscope 12.

The tube member 62 is formed by bending it into a ‘U’ or ‘J’ shape. According to this configuration, it is possible to realize a tube member 62 that can locate the flexible tube 55 in the proximity of the endoscope 12 with a very simple configuration.

The holding portion 56 is provided in such a manner that it is detachable from the insertion portion 17 of the endoscope 12. According to this configuration, the surgeon can perform an advance/retreat operation of the treatment device 13 with the fingers of their right hand, while gripping the insertion portion 17 with their right hand. Thus, it is possible to prevent the flexible tube 55 from interfering with a surgeon's and assistant's work and to improve workability of the surgeon.

The holding portion 56 is provided at the end portion of the flexible tube 55. According to this configuration, the end portion of the flexible tube 55 does not come off from the insertion portion 17 or the operation portion 16 of the endoscope 12, thereby preventing the flexible tube 55 from interfering with the surgeon's or the assistant's work.

### Second Embodiment

Subsequently, the second embodiment of the endoscope system 11 will be explained with reference to FIG. 4. The endoscope system 11 of the second embodiment differs from that of the first embodiment in terms of the shape of the tube member employed, but the other features are similar to those of the first embodiment. Therefore, mainly the parts different from those of the first embodiment will be described in the following, and illustrations or descriptions of the parts identical to those of the first embodiment will be omitted.

The tube member 62 is made from a cylindrical tube made of a resin or a metal, for example. The tube member 62 has strength to the extent that it is not deformed under a certain level of externally-applied power, and thus, the tube member 62 can be regarded as almost a rigid body. In other words, the tube member 62 has higher rigidity or is harder than the flexible tube 55. The tube member 62 includes an end 62A coupled to the coupling portion 45, an other end 62B coupled to the flexible tube 55, and a curved portion 62C provided in between in the longitudinal direction. The second central axis C2 of the end 62B extends in a direction different from the direct on of the first central axis C1 of the end 62A. In the present embodiment, the second central axis C2 of the end 62B extends in a direction along the direction of the third central axis C3, which is the central axis of the operation portion 16 and the insert on portion 17.

The tube member 62 is in an overall ‘U’ or a question mark (“?”) shape. The radius of curvature of the curved portion 62C of the tube member 62 is appropriately determined to be of a value as small as possible within a range that allows the treatment device 13 to advance and retreat with no problems. Thus, in the present embodiment, since the radius of curvature of the tube member 62 is formed as minimal as possible, the tube member 62 does not interfere when handling the operation portion 16, as the tube member 62 does not project from the operation portion 16 and the insertion portion 17. The inner surface of the tube member 62 (the insertion path 58) is coated with a fluorine-based resin to provide excellent slidability for the treatment device 13 in the insertion path 58.

The effects of the endoscope system 11 according to the present embodiment are approximately the same as those of the first embodiment. In other words, the surgeon can perform delicate techniques of high-frequency incision with, for example, the treatment device 13 using high-frequency energy, by delicately adjusting the length of the projection of the end effector 52 of the treatment device 13 from the distal end constituent portion 21 of the insertion portion 17 of the endoscope 12 with the thumb and first finger of their right hand, while performing a push-and-pull operation and a twist operation of the insertion portion 17, gripping the insertion portion 17 with their right hand. At this time, since the tube member 62 causes the flexible tube 55 to be arranged in the proximity of the insertion portion 17 and the operation portion 16 of the endoscope 12, the flexible tube 55 does not interfere with the surgeon's and operator's work.

According to the present embodiment, the tube member 62 includes an end 62A coupled to the coupling portion 45 and the end 62B coupled to the flexible tube 55, and in which the central axis extends in the direction of the central axis of the insertion portion 17 of the endoscope 12. According to this configuration, it is possible to control the position of the flexible tube 55 in such a manner that the flexible tube 55 is arranged along the insertion portion 17 due to the tube member 62. Thus, it is possible to prevent the flexible tube 55 from interfering with a surgeon's and operator's work and to improve workability of the surgeon and the operator.

### Third Embodiment

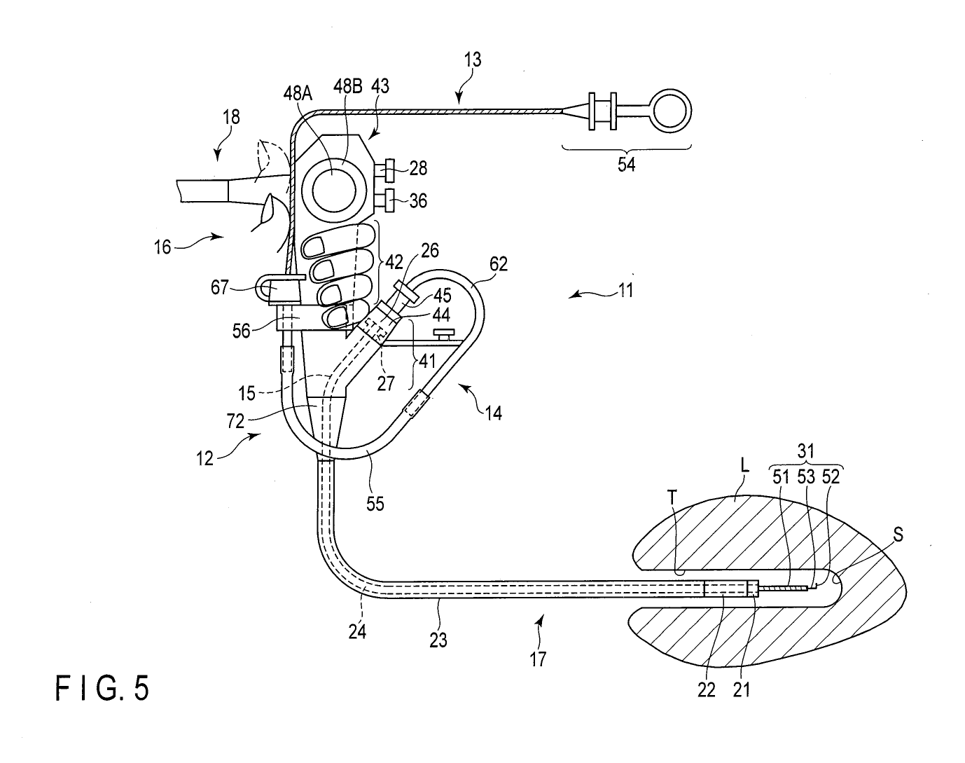

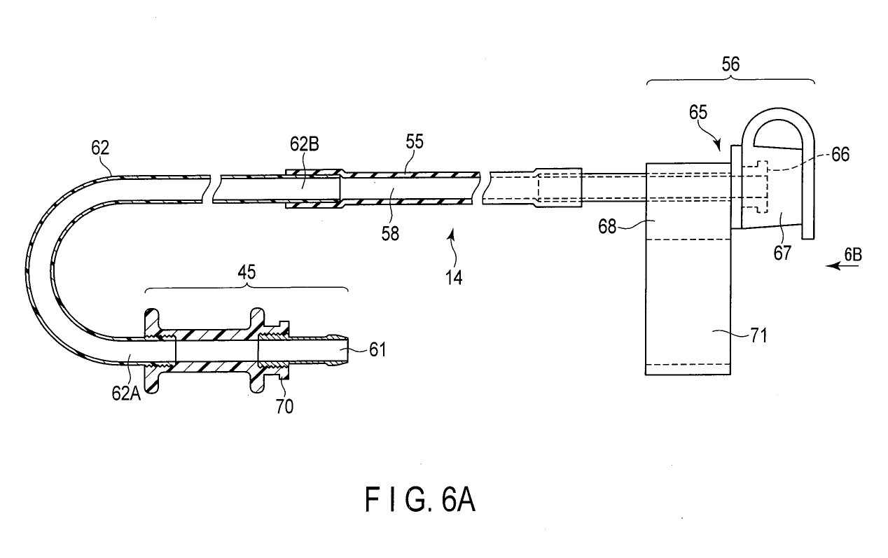

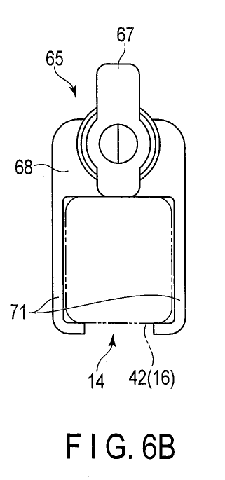

Subsequently, the third embodiment of the endoscope system 11 will be explained with reference to FIGS. 5, 6A, and B. The endoscope system 11 of the third embodiment differs from that of the first embodiment in terms of the structure of the holding portion 56 of the assistance tool 14 that assists in moving the treatment device and of the position where the holding portion 56 is arranged with respect to the endoscope 12, but the other features are similar to those of the first embodiment. Therefore, mainly the parts different from those of the first embodiment will be described in the following, and illustrations or descriptions of the parts identical to those of the first embodiment will be omitted.

As shown in FIG. 5, the holding portion 56 can hold the outer surface of the operation portion 16 of the endoscope 12 on the distal end (a moving end) side of the flexible tube 55. The holding portion 56 is arranged in the proximity of the operation portion 16 (the grip portion 42), i.e., in the proximity of the thumb of the surgeon s left hand. For this reason, the holding portion 56 can determine the position of the flexible tube 55 in such a manner that the other end of the flexible tube 55 is directed in a direction toward the thumb of the surgeon's left hand.

As shown in FIGS. 5, 6A, and 6B, the holding portion 56 includes a holding portion main body 65, a third coupling base 66 attached to the holding portion main body 65, and a treatment device plug 67. The third coupling base 66 has a same mechanism as that of the first coupling base 27 of the endoscope 12. Similar to the above-described assistance tool plug 44, a plug similar to a publicly-known forceps plug may be used as a treatment device plug 67. Thus, the treatment device plug 67 can prevent body fluid in the body cavity flowing in the treatment device insertion channel 15 of the endoscope 12 and the insertion path 58 of the assistance tool 14 from leaking out because of its valve structure. Furthermore, even when the insertion body 31 of the treatment device is being inserted, the treatment device plug 67 can prevent liquid, such as body fluid in the body cavity flowing in the treatment device insertion channel 15 of the endoscope 12 and the insertion path 58 of the assistance tool 14, from leaking out. The treatment device plug 67 serves as a brake that prevents the treatment device 13 from being moved unintentionally.

The holding portion main body 65 is made of a resin, etc., for example. The holding portion main body 65 includes a base body 68 that forms the proximal end portion of the insertion path 58 and a pair of arms 71 extending from the base body 68. The pair of arms 71 forms a frame-like shape partially having an open portion (a release portion). In other words, the pair of arms 71 makes a ‘C’ shape. The holding portion main body 65 can hold the grip portion 42 between the arms 71 when the space between the arms is temporarily widened for the operation portion 16 (the grip portion 42), which is shown in a dashed line, as a result of elastically deforming the arms 71. Thus, the holding portion main body 65 can be easily attached to and detached from the outer surface of the grip portion 42. The pair of arms 71 can hold not only the grip portion 42, but also the bend prevention portion 72 located at the proximal end side of the insertion portion 17.

The effects of the endoscope system 11 according to the present embodiment are approximately the same as those of the first embodiment. In other words, the surgeon can perform delicate techniques of high-frequency incision with, for example, the treatment device 13 using high-frequency energy, by delicately adjusting the length of the projection of the end effector 52 of the treatment device 13 from the distal end constituent portion 21 of the insertion portion 17 of the endoscope 12 with the thumb and first finger of their left hand, while performing a push-and-pull operation and a twist operation of the insertion portion 17, gripping the insertion portion 17 with their right hand. Similarly, the surgeon moves the treatment device 13 up and down, or left and right by pressing the treatment device 13 against the outer surface of the operation portion 16 by their left thumb to advance/retreat or rotate the treatment device 13.

At this time, the tube member 62 and the holding portion 56 attached to the grip portion 42 cause the flexible tube 55 to be arranged in the proximity of the insertion portion 17 and the operation portion 16 of the endoscope 12, and the flexible tube 55 does not interfere with the surgeon's and operator's work.

According to the third embodiment, since the position of the flexible tube 55 can be arranged in the proximity of the endoscope 12, particularly in the proximity of the operation portion 16 of the endoscope 12, by the tube member 62 and the holding portion 56 fixed to the grip portion 42, it is possible to improve workability of the surgeon's and operator's work.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An assistance tool that assists in moving a treatment device, comprising:
a flexible tube in which an insertion path is formed to insert the treatment device that is inserted into a channel in an insertion portion of an endoscope;
a tube member that is coupled to the flexible tube and has a curved portion halfway of an overall length and in which an insertion path for the treatment device is formed by a rigid tube;
a coupling portion that is coupled to the tube member and is formed to couple the channel to the insertion path of the tube member; and
a holding portion that is attached to the flexible tube and holds the insertion portion of the endoscope or an operation portion of the endoscope that is coupled to the insertion portion.

2. The assistance tool that assists in moving a treatment device according to claim 2, wherein the tube member is formed by bending it into a ‘U’ or ‘J’ shape.

3. The assistance tool that assists in, moving a treatment device according to claim 1, wherein the holding portion is detachably provided in the insertion portion of the endoscope.

4. The assistance tool that assists in moving a treatment device according to claim 1, wherein the holding portion is provided at an end portion of the flexible tube.

5. An endoscopy system comprising:
the assistance tool according to claim 1;
the endoscope including the insertion portion that is inserted into a body cavity and in which the channel for inserting a treatment device into an inside thereof is provided, and the operation portion that is coupled to the insertion portion and operates the insertion portion; and
the treatment device that is inserted into the endoscope and is assisted by the assistance tool to move.
